(19) United States
(12) Patent Application Publication
(10) Pub. No.: US 2008/0299046 A1
(43) Pub. Date: Dec. 4, 2008

(54) METHODS FOR CONTROLLING SURFACE FUNCTIONALITY OF METAL OXIDE NANOPARTICLES, METAL OXIDE NANOPARTICLES HAVING CONTROLLED FUNCTIONALITY, AND USES THEREOF

(75) Inventors: Meghann A. White, New York, NY (US); Jeffrey T. Koberstein, Storrs, CT (US); Nicholas J. Turro, Tenafly, NJ (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/873,163

(22) Filed: Oct. 16, 2007

Related U.S. Application Data
(60) Provisional application No. 60/852,157, filed on Oct. 16, 2006.

(51) Int. Cl. A61K 49/18; C07F 15/02; A61K 9/14; C09D 1/00; B05D 5/12; A61P 35/00; C07F 7/28

(52) U.S. Cl. 424/9.32; 556/146; 556/54; 424/501; 106/287.35; 427/126.3

(57) ABSTRACT

Methods for controlling surface functionality of metal oxide nanoparticles, nanoparticles having controlled surface functionality, and uses thereof are described herein. Methods for controlling the surface functionality of a metal oxide nanoparticle are can include attaching a ligand to a metal oxide nanoparticle, where the ligand can include a functional portion that is capable of forming an irreversible bond with an object at a site that is complementary to the functional portion without reacting with other reactive sites that may be present. Moreover, metal oxide nanoparticles having versatile ligands can include an anchoring portion that binds to the surface of the metal oxide nanoparticle and a functional portion that is capable of forming an irreversible bond with an object at a site that is complementary to the functional portion without reacting with other reactive sites that may be present. Uses thereof can include cancer detection, electronics, cosmetics, cellular delivery carriers, magnetic storage media, drug delivery carriers, nanocomposite formation for improved mechanical properties, and the like.

### CROSS-REFERENCE TO RELATED APPLICATIONS

The present application claims the benefit of the filing date of U.S. Patent Application No. 60/852,157, filed on Oct. 16, 2006, the content of which is hereby incorporated by reference herein in its entirety.

### STATEMENT REGARDING FEDERALLY SPONSORED RESEARCH OR DEVELOPMENT

The invention was made with United States government support under Grant No. RFCUNY #404340001A awarded by the National Science Foundation (NSF) through the Integrative Graduate Education and Research Traineeship (IGERT). The United States government may have certain rights in this invention.

### COPYRIGHT NOTICE

This patent disclosure may contain material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### INCORPORATION BY REFERENCE

All patents, patent applications and publications cited herein are hereby incorporated by reference in their entirety. These disclosures in their entireties are hereby incorporated by reference into this application in order to more fully describe the state of the art as known to those skilled therein as of the date of the invention described herein.

### FIELD OF THE INVENTION

The invention relates to methods for controlling surface functionality of metal oxide nanoparticles, metal oxide nanoparticles having controlled surface functionality, and uses thereof.

### BACKGROUND OF THE INVENTION

Metal oxide nanoparticles have recently gained attention for their potential uses in various applications. However, these nanoparticles are compatible with only certain types of materials and cannot easily be employed in a wide variety of applications. To overcome these problems, ligands have been attached on the nanoparticles to allow compatibility with the environment. As one particular ligand is not suitable for all the various different environments, ligand exchange has been developed, where ligands on the nanoparticle surface are stripped off and functionalized with other suitable ligands.

However, a significant drawback exists to this approach because ligands are not strongly bound to the nanoparticle surface to facilitate the ligand exchange. Over time (e.g., during storage), these ligands can become unbound from the nanoparticle surface and aggregation of the nanoparticles can occur.

Therefore, there is currently a need for ligands that are robust and that can be readily tailored for different and specific types of applications.

### SUMMARY OF THE INVENTION

The invention provides methods for controlling surface functionality of metal oxide nanoparticles, metal oxide nanoparticles having controlled surface functionality, and uses thereof. The invention provides a strategy for the synthesis of surface functionalized metal oxide nanoparticles through the design of versatile ligands.

Methods for controlling the surface functionality of a metal oxide nanoparticle are provided. Such methods can include attaching a ligand to a metal oxide nanoparticle, where the ligand can include a functional portion that is capable of forming an irreversible bond with an object at a site that is complementary to the functional portion.

Metal oxide nanoparticles having versatile ligands are provided. The versatile ligands can include an anchoring portion that binds to the surface of the metal oxide nanoparticle and a functional portion that is capable of forming an irreversible bond with an object only at a site that is complementary to the functional portion.

Methods for detecting cancer cells are provided. Such methods can include attaching a ligand to a metal oxide nanoparticle, where the ligand has a functional portion that is capable of forming an irreversible bond with a marker that has an affinity to cancer cells. Methods of the invention can further include reacting the ligand with the marker to form a metal oxide nanoparticle having an affinity for cancer cells, administering a sufficient quantity of the nanoparticle having an affinity for cancer cells to a patient in need thereof, and detecting the cancer cells through magnetic resonance imaging.

Methods for forming a high dielectric constant material in electronic devices are also provided. Such methods can include attaching a ligand to a metal oxide nanoparticle having a high dielectric constant, where the ligand can include a functional portion that is capable of forming an irreversible bond with a resin only at a site that is complementary to the functional portion. The resin can be compatible with electronic device manufacturing requirements. Methods of the invention can further include reacting the ligand with the resin to form a metal oxide nanoparticle that is compatible with electronic device manufacturing requirements and depositing the metal oxide nanoparticle that is compatible with electronic device manufacturing requirements onto at least a portion of an electronic device.

### BRIEF DESCRIPTION OF THE DRAWINGS

The above and other objects and advantages of the invention will be apparent upon consideration of the following detailed description, taken in conjunction with the accompanying drawings, in which like reference characters refer to like parts throughout, and in which:

FIG. 1 is a diagram illustrating controlling surface functionality of metal oxide nanoparticle using click chemistry.

FIG. 2 is a diagram illustrating a chemical reaction for controlling surface functionality of iron oxide nanoparticles using a phosphonic acid-azide ligand undergoing a 1,3-dipolar cycloaddition with 5-chloropentyne.

FIG. 2A is a TEM image of iron oxide nanoparticles having phosphonic acid-azide ligand.

FIG. 2B is an FTIR spectrum of phosphonic acid-azide ligand bound to iron oxide nanoparticles and unbound phosphonic acid-azide ligand.

FIG. 2C is an FTIR spectra of iron oxide nanoparticles having phosphonic acid-azide ligand, iron oxide nanoparticles having phosphonic acid-azide ligand that has undergone 1,3-dipolar cycloaddition with 5-chloropentyne, and 2-[4-(3-chloro-propyl)-[1,2,3]triazol-1-yl]-2-methyl-propionic acid 2-hydroxy-ethyl ester.

FIG. 2D is an NMR spectra of iron oxide nanoparticles having phosphonic acid-azide ligand that have undergone 1,3-dipolar cycloaddition with 5-chloropentyne and 2-[4-(3-chloro-propyl)-[1,2,3]triazol-1-yl]-2-methyl-propionic acid 2-hydroxy-ethyl ester.

FIG. 2E is a TEM image of iron oxide nanoparticles having phosphonic acid-azide ligand that have undergone 1,3-dipolar cycloaddition with 5-chloropentyne.

FIG. 3 is a diagram illustrating controlling surface functionality of iron oxide nanoparticles using a 5-hexynoic acid ligand functionalized with benzyl azide.

FIG. 3A is a TEM image of iron oxide nanoparticles having 5-hexynoic acid ligand.

FIG. 3B is an FTIR spectra of 5-hexynoic acid ligand bound to iron oxide nanoparticles and unbound 5-hexynoic acid ligand.

FIG. 3C is an FTIR spectra of iron oxide nanoparticles having 5-hexynoic acid ligand, iron oxide nanoparticles having 5-hexynoic acid ligand that have undergone 1,3-dipolar cycloaddition with benzyl azide, and 4-(1-benzyl-1H-[1,2,3]triazol-4-yl)-butyric acid.

FIG. 3D is an NMR spectra of iron oxide nanoparticles having 5-hexynoic acid ligand that have undergone 1,3-dipolar cycloaddition with 5-chloropentyne and 4-(1-benzyl-1H-[1,2,3]triazol-4-yl)-butyric acid.

FIG. 3E is a TEM image of iron oxide nanoparticles having 5-hexynoic acid ligand that have undergone 1,3-dipolar cycloaddition with benzyl azide.

FIG. 4 is a diagram illustrating a chemical reaction for controlling surface functionality of iron oxide nanoparticles using a phosphonic acid ligand that have undergone 1,3-dipolar cycloaddition with α-acetylene poly(tert-butyl acrylate) (ptBA) polymer.

FIG. 4A is an FTIR spectrum of α-acetylene-poly(tert-butyl acrylate (ptBA) and iron oxide nanoparticles having phosphonic acid-azide ligand that have undergone 1,3-dipolar cycloaddition with ptBA.

FIG. 4B is an NMR spectrum of iron oxide nanoparticles having phosphonic acid-azide ligand that have undergone 1,3-dipolar cycloaddition with ptBA.

FIG. 4C is a TEM image of iron oxide nanoparticles that have undergone 1,3-dipolar cycloaddition with poly(t-butyl acrylate).

FIG. 5 is a diagram illustrating a chemical reaction for controlling surface functionality of titanium dioxide nanoparticles using a dodec-11-ynyl-phosphonic acid diethyl ester ligand and undergoing a 1,3-dipolar cycloaddition with ω-azido polystyrene polymer.

FIG. 5A is a TEM of titanium dioxide nanoparticles having a dodec-11-ynyl-phosphonic acid diethyl ester ligand.

FIG. 5B is a TEM image of titanium dioxide nanoparticles that have undergone 1,3-dipolar cycloaddition with ω-azido polystyrene polymer.

FIG. 6 is a diagram illustrating a chemical reaction for controlling surface functionality of titanium dioxide nanoparticles using a dodec-11-ynyl-phosphonic acid diethyl ester ligand and undergoing a 1,3-dipolar cycloaddition with ω-azido poly(tert-butyl acrylate) polymer.

FIG. 6A is a TEM image of titanium dioxide nanoparticles that have undergone 1,3-dipolar cycloaddition with ω-azido poly(tert-butyl acrylate).

FIG. 7 is a plot of dielectric constant measured from films of nanoparticles that have undergone 1,3-dipolar cycloaddition with ω-azido poly(tert-butyl acrylate) or ω-azido polystyrene.

### DETAILED DESCRIPTION OF THE INVENTION

The invention provides methods for obtaining surface functionalized metal oxide nanoparticles through the design of versatile ligands. In some embodiments, versatile ligands can include an anchor portion that can bind to a variety of metal oxide surfaces and a functional portion that can be attached to other objects. In certain embodiments, the functional portion can be positioned at the perimeter of the versatile ligand (i.e., near the end of the versatile ligand that is away from the nanoparticle surface). In other embodiments, versatile ligands of the invention can further include portions that act as spacers between the anchor portion and the functional portion.

As used herein, the term “versatile ligand” refers to a group or a molecule that includes at least an anchor that can be attached to a surface of metal oxide nanoparticles and a functional portion that can be attached to other objects.

The functional portion of the versatile ligand can allow attachment of other objects to the versatile ligand. Numerous different objects can be attached to the functional perimeter. Some examples of such objects include atoms, molecules, proteins, viruses, polymers, and the like, which can be attached to the functional perimeter portion of the versatile ligand. For example, attachment can be carried out by forming suitable bonds (e.g., covalent, ionic, etc.) between the object and the functional portion. Other nanoparticles, with or without ligands, can also be bound to the versatile ligands. The functional portion of the versatile ligands can also be attached to any desired surfaces, whether the surface is flat, curved, etc.

In certain embodiments, the functional portion can form a covalent bond with another molecule through click chemistry. As used herein, “click chemistry” refers to reactions that have at least the following characteristics: (1) exhibits functional group orthogonality (i.e., the functional portion reacts only with a reactive site that is complementary to the functional portion, without reacting with other reactive sites); and (2) the resulting bond is irreversible (i.e., once the reactants have been reacted to form products, decomposition of the products into reactants is difficult). Optionally, click chemistry can further have one or more of the following characteristics: (1) stereospecificity; (2) reaction conditions that do not involve stringent purification, atmospheric control, and the like; (3) readily available starting materials and reagents; (4) ability to utilize benign or no solvent; (5) product isolation by crystallization or distillation; (6) physiological stability; (7) large thermodynamic driving force (e.g., 10-20 kcal/mol); (8) a single reaction product; (9) high (e.g., greater than 50%) chemical yield; and (10) substantially no byproducts or byproducts that are environmentally benign byproducts.

Examples of click chemistry can include, but are not limited to, addition reactions, cycloaddition reactions, nucleophilic substitutions, and the like. Examples of cycloaddition reactions can include Huisgen 1,3-dipolar cycloaddition, Cu(I) catalyzed azide-alkyne cycloaddition, and Diels-Alder reactions. Examples of addition reactions include addition reactions to carbon-carbon double bonds such as epoxidation and dihydroxylation. Nucleophilic substitution examples can include nucleophilic substitution to strained rings such as epoxy and aziridine compounds. Other examples can include formation of ureas and amides. Some additional description of click chemistry can be found in Huisgen, Angew. Chem. Int. Ed., Vol. 2, No. 11, 1963, pp. 633-696; Lewis et al., Angew. Chem. Int. Ed., Vol. 41, No. 6, 2002, pp. 1053-1057; Rodionov et al., Angew. Chem. Int. Ed., Vol. 44, 2005, pp. 2210-2215; Punna et al., Angew. Chem. Int. Ed., Vol. 44, 2005, pp. 2215-2220; Li et al., J. Am. Chem. Soc., Vol. 127, 2005, pp. 14518-14524; Himo et al., J. Am. Chem. Soc., Vol. 127, 2005, pp. 210-216; Noodleman et al., Chem. Rev., Vol. 104, 2004, pp. 459-508; Sun et al., Bioconjugate Chem., Vol. 17, 2006, pp. 52-57; and Fleming et al., Chem. Mater., Vol. 18, 2006, pp. 2327-2334, the contents of which are hereby incorporated by reference herein in their entireties.

In certain embodiments, anchor portion of the versatile ligand can include any atom or group that can bind to the surface of metal oxide nanoparticles. Anchors that can bind to a metal oxide surface can include carboxylates, alcohols, phosphonates, phosphonic acid esters, siloxanes, enediols, diols, catechol, and the like. For example, ligands having anchors that can bind to a metal oxide surface can include trioctylphosphine oxide, myristic acid, caprylic acid, 2-bromo-2-methylpropionic acid, dodecanol, 2,2′-didodecyl-1,3-dihydroxypropane, 2-dodecyl-1,3-dihydroxypropane, tridodecylcarbinol, didodecylcarbinol, octadecylphosphonic acid, lauric acid-liposome, trioctylamine, octylamine, dodecylamine, hexadecylamine, oleylamine, octanethiol, dodecanethiol, and the like.

In certain embodiments, metal oxide nanoparticles can be synthesized to include the versatile ligands. In other embodiments, other ligands that exist on metal oxide nanoparticle surfaces can be replaced with the versatile ligands. For example, metal oxide nanoparticles having other ligands, such as oleic acid, can be made (see, e.g., Maliakal, A., Katz, H., Cotts, P. M., Subramoney, S., Mirau, P. J. Am. Chem. Soc. 2005, vol. 127, p. 14655; and Yin, M., Willis, A., Redl, F. Turro, N. J., O'Brien, S. P., J. Mater. Res. 2004, vol. 19, p. 1208; both of which are hereby incorporated by reference herein in their entireties). Such metal oxides having other ligands can be subjected to an exchange reaction to replace the other ligands with the versatile ligands.

Numerous different metal oxide nanoparticles having the versatile ligands are provided by the invention. For example, iron oxide (e.g., Fe₂O₃, Fe₃O₄) titanium oxide (e.g., anatase, rutile), silicon oxide (e.g., SiOₓ), aluminum oxide (e.g., Al₂O₃), vanadium oxide (e.g., V₂O₅), copper oxide (e.g., CuO, Cu₂O), cobalt oxide (e.g., CoO), manganese oxide (Mn₃O₄), zinc oxide (e.g., ZnO), europium oxide (e.g., Eu₂O₃), gadolinium oxide (e.g., Gd₂O₃), indium oxide (e.g., In₂O₃), barium titanium oxide (e.g., BaTiO₃), manganese iron oxide (e.g., MnFe₂O₄), cobalt iron oxide (e.g., CoFe₂O₄), nickel iron oxide (e.g., NiFe₂O₄), zinc iron oxide (e.g., ZnFe₂O₄) and the like can be modified with the versatile ligands.

The metal oxide nanoparticles can be of any shape, such as spherical, rod-like, plate-like, ellipsoidal, hemispherical, hemi ellipsoidal, tripod-like, tetrapod-like, and the like.

The metal oxide nanoparticles can include nanoparticles that are less than 1000 nanometers in size, less than 500 nanometers in size, less than 200 nanometers in size, less than 100 nanometers in size, less than 50 nanometers in size, less than 25 nanometers in size, less than 20 nanometers in size, less than 10 nanometers in size, less than 5 nanometers in size, and even less than 1 nanometer in size. However, the metal oxide nanoparticles are generally larger than the size of the anchor of the versatile ligand. In certain embodiments, metal oxide nanoparticles can be from about 10 nanometers to about 100 nanometers.

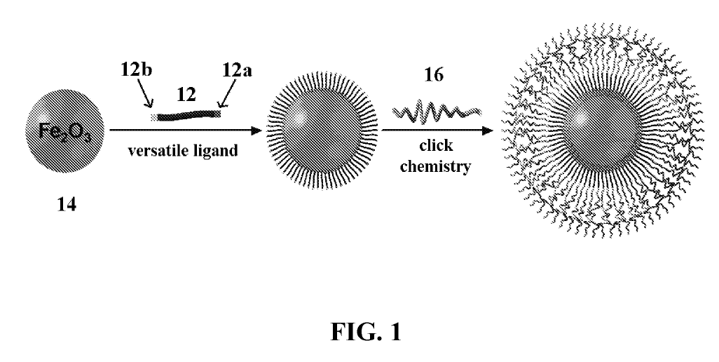

FIG. 1 shows an example where a versatile ligand 12 can be attached to a metal oxide nanoparticle 14 (Fe₂O₃ nanoparticle shown in FIG. 1) through an anchor portion 12a, followed by attachment of an object 16 (a molecule shown in FIG. 1) to the functional portion 12b of the versatile ligand (“click” chemistry shown in FIG. 1).

### APPLICATIONS

Metal oxide nanoparticles having the versatile ligands described above can be useful in a number of different applications.

Metal oxide nanoparticles having versatile ligands can be utilized in cosmetic applications. For example, titanium dioxide or zinc oxide nanoparticles (or other nanoparticles that can absorb ultraviolet radiation) can be utilized as sunscreens. The versatile ligands can be utilized to functionalize the metal oxide nanoparticles with suitable molecule(s) to allow the metal oxide nanoparticles to form a stable dispersion in a desired medium (e.g., water). In other embodiments, phase transfer reaction can be utilized to convert the functionalized molecule(s) to have desired properties. For example, titanium dioxide nanoparticles having versatile ligands can be functionalized with poly(tert-butyl acrylate) (ptBA) using click chemistry, which may be able to form stable dispersions in nonpolar solvents. Subsequently, a phase transfer reaction can be carried out where ptBA can be converted into polyacrylic acid (PAA) and allow the metal oxide nanoparticles to form stable dispersion in water.

Metal oxide nanoparticles having the versatile ligand can further be utilized in paint applications. For example, certain nanoparticles are clear due to its sub-optical wavelength sizes. However, if theses nanoparticles agglomerate, the agglomerates can begin to interact with the optical wavelengths and scatter light, causing opacity. Therefore, metal oxide nanoparticles can be suitably functionalized to remain substantially agglomerate-free and optically clear. Such metal oxide nanoparticles can be included paint formulations to improve desired properties (e.g., mechanical properties). In some embodiments, metal oxide nanoparticles capable of absorbing ultraviolet radiation can be applied as coatings to protect an underlying paint layer. For example, titanium oxide or zinc oxide nanoparticles can be functionalized with a desired polymer and applied as a clearcoat to automobiles to protect the underlying paint finishes.

Metal oxide nanoparticles having versatile ligands can be employed in various medical applications. For example, iron oxide materials are useful as contrast agents in magnetic resonance imaging. Therefore, the possibility of placing such contrast agents in specific locations in the body would be beneficial. To illustrate, iron oxide nanoparticles having the versatile ligands can be functionalized with molecules that have an affinity for tumors such as tumor specific markers (e.g., antibodies that can detect antigens only found on cancer cells). Such nanoparticles can be utilized for magnetic resonance imaging contrast markers to preferentially locate cancerous cells in a body.

Another example for utilizing metal oxide nanoparticles having versatile ligands in medical applications can include attaching a bifunctional molecule (e.g., α-ω heterobifunctional polymer) to the versatile ligand. One functional portion of the bifunctional molecule can react with the versatile ligand (e.g., through click chemistry) and the other functional portion of the bifunctional polymer can be utilized to conjugate with a biological molecule having affinity for specific cells, tissues, and the like. To illustrate, iron oxide nanoparticles having versatile ligands can be functionalized with ptBA using click chemistry, where the ptBA has a terminal portion that can further be reacted. Subsequently, ptBA can be converted to polyacrylic acid (PAA) to render the polymer hydrophilic. The terminal portion of the PAA can be functionalized with a molecule that has an affinity for tumors, such as anti-vascular endothelial growth factor (anti-VEGF). (VEGF is a protein that is involved in angiogenesis—the growth of blood vessels in tumors). The iron oxide nanoparticles having anti-VEGF molecules can then be administered to selectively bind to the tumor cells. Magnetic resonance imaging can be utilized to confirm that the iron oxide nanoparticles are attached to the tumors and magnetic heating can be utilized to heat and kill the tumors.

Metal oxide nanoparticles having versatile ligands can also be employed as drug delivery devices. For example, the metal oxide nanoparticles can be functionalized with a block copolymer wherein one block is hydrophobic and another block is hydrophilic. The block copolymer can be attached to the metal oxide nanoparticles via click chemistry through the versatile ligands. In some embodiments, the hydrophobic block can be attached to the versatile ligand and the hydrophilic block can form a shell around the hydrophobic block. The hydrophobic block can be capable of carrying suitable drugs and the hydrophilic block can be biocompatible and dispersible in the bloodstream. The block copolymer can be formed utilizing any suitable methods, such as ATRP, anionic polymerization, cationic polymerization, reversible addition fragmentation chain transfer polymerization, and the like. Some suitable hydrophobic blocks of the block copolymer can include polystyrene, poly(tert-butyl methacrylate), poly(methyl methacrylate), polybutadiene, polyisoprene, fluorinated acrylate polymers, and the like. Some suitable hydrophilic blocks can include polyacrylic acid, poly(hydroxy ethyl methacrylate), poly(hydroxylethyl acrylate), polyvinyl pyridine, and the like. Such nanoparticles carrying desired drugs can be administered to a patient as a time-release drug.

Metal oxide nanoparticles having versatile ligands can be employed in various electronics applications. For example, titanium dioxide exhibits a high dielectric constant of about 30 to 100, depending on the crystal form (i.e., anatase or rutile). Utilization of titanium dioxide nanoparticles (or any other metal oxide nanoparticles with high dielectric constant from about 10 to about 300) having versatile ligands can be beneficial in obtaining high dielectric constant material that can be easily patterned onto desired locations. To illustrate, titanium dioxide nanoparticles having the versatile ligands can be functionalized with polymers that are compatible with electronic device manufacturing specifications, such as polymethyl methacrylate (PMMA). Such titanium dioxide nanoparticles functionalized with a matrix polymer can be deposited and electron beam (or lithographically) patterned during electronic device manufacturing operations. Utilizing PMMA as an exemplary matrix polymer, the depolymerization of the matrix polymer exposed to radiation can facilitate removal in areas that are not required. Alternatively, the functionalized nanoparticles can be printed (e.g., ink-jet printed). Some other high dielectric metal oxide nanoparticles can include BaTiO₃, Al₂O₃, Gd₂O₃, Yb₂O₃, Dy₂O₃, Nb₂O₅, Y₂O₃, La₂O₃, ZrO₂, HfO₂, Ta₂O₅, SrTiO₃, BaₓSr₁₋ₓTiO₃, ZrₓSi₁₋ₓO_{y}, HfₓSi₁₋ₓO_{y}, AlₓZr₁₋ₓO₂ Pr₂O₃, and the like.

In some other embodiment, nanoparticles having first versatile ligands and nanoparticles having second versatile ligands, where the first and second versatile ligands have complementary functional portions to each other for carrying out click chemistry, can be reacted to provide a homogeneous distribution of nanoparticles in a film. The click chemistry reaction can provide a film that has a uniform and precisely defined spacing between the nanoparticles to prevent agglomeration. Such uniform distribution of nanoparticles may further be beneficial in obtaining consistently uniform film roughness and dielectric constant. This can be beneficial because if the nanoparticles agglomerate, there may be regions of high dielectric constant and low dielectric constant. In that case, variability of device (e.g., transistors) performance may arise between areas having agglomerated nanoparticles and lesser amount of nanoparticles. Therefore, the nanoparticles of the invention can allow more consistent device performance over a large number of devices.

Numerous other applications, such as cellular delivery carriers, magnetic storage media, nanocomposite formation for improved mechanical properties, and the like can be mentioned. As described, numerous potential applications and uses can be envisioned, as will be readily apparent to one of ordinary skill in the art.

### EXAMPLES

### Example 1

γ-Fe₂O₃ nanoparticles having oleic acid ligands were synthesized as described in Yin, M., Willis, A., Redl, F. Turro, N. J., O'Brien, S. P., J. Mater. Res. 2004, 1 vol. 9, p. 1208. The nanoparticles are crystalline and well dispersed and have about less than 5% rms variation in size.

A versatile ligand was synthesized as follows. Anhydrous ethylene glycol (225 mL, 4.1 mol) was added to a 500 mL 2-neck round bottom flask that had been flame-dried under vacuum and purged three times with argon. The flask was equipped with a magnetic stir bar and rubber septum. The flask was then cooled to 0° C. for 3 hours. The reaction was quenched with 100 mL H₂O and extracted with CHCl₃ (3×100 mL). The combined organic extracts were dried over MgSO₄, filtered, and the CHCl₃ was removed by a rotary evaporator. The subsequent liquid was purified by distillation (85° C., 30 mTorr) to yield 2-bromo-2-methyl-propionic acid 2-hydroxy-ethyl ester as a viscous, clear, colorless liquid (30.4 g, 89%). Then, 2-bromo-2-methyl-propionic acid 2-hydroxy-ethyl ester (2.0 g, 9.48 mmol) was dissolved in anhydrous DMF (15 mL) in a 2-neck round bottom flask that had been flame-dried and purged with argon 3 times. The flask was equipped with a magnetic stir bar and rubber septum. NaN₃ (677 mg, 10.42 mmol) was then added to the stirring solution. The reaction stirred at ambient temperature (21° C.) for 20 hours. The reaction was quenched with H₂O (20 mL) and extracted with CHCl₃ (3×20 mL). The combined organic extracts were filtered over MgSO₄ and the solvent was removed by a rotary evaporator. The resultant liquid was dried under vacuum overnight to remove additional DMF. This yielded 2-azido-2-methyl-propionic acid 2-hydroxy-ethyl ester as a clear, colorless liquid (1.51 g. 94%) that required no further purification. Then, 2-azido-2-methyl-propionic acid 2-hydroxy-ethyl ester (1.00 g, 5.77 mmol) was dissolved in anhydrous THF (15 mL) in a flame-dried 2-neck round bottom flask that was purged three times with argon. The flask was equipped with a magnetic stir bar and rubber septum. Anhydrous triethylamine (0.9 mL, 6.35 mmol) was added, and the mixture was cooled to 0° C. with an ice bath. POCl₃ (0.6 mL, 6.35 mmol) was added drop wise to the cooled solution. The mixture became cloudy and white upon addition of the POCl₃. The reaction mixture was allowed to warm to ambient temperature (21° C.) as the ice melted and stirred for 3 additional hours. The reaction became light yellow and clear, at which point the reaction was quenched with H₂O (10 mL). The pH was checked to ensure it was less than 2 and extraction was carried out with CHCl₃ (3×15 mL). The combined organic extracts were filtered over MgSO₄ and the solvent was removed from the product using a rotary evaporator. The resultant liquid was dried for several hours under vacuum (10 mTorr) to remove excess solvent. This yielded 2-azido-w-methyl-propionic acid 2-phosphonooxy-ethyl ester (hereinafter “phosphonic acid-azide ligand”) as a light amber, highly viscous, clear liquid (0.80 g, 55%).

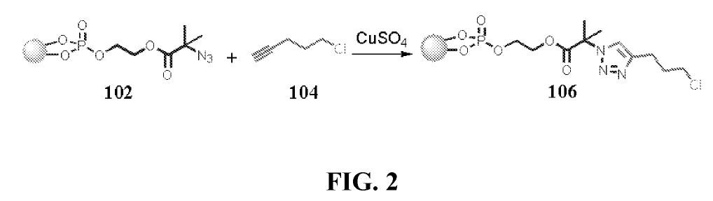

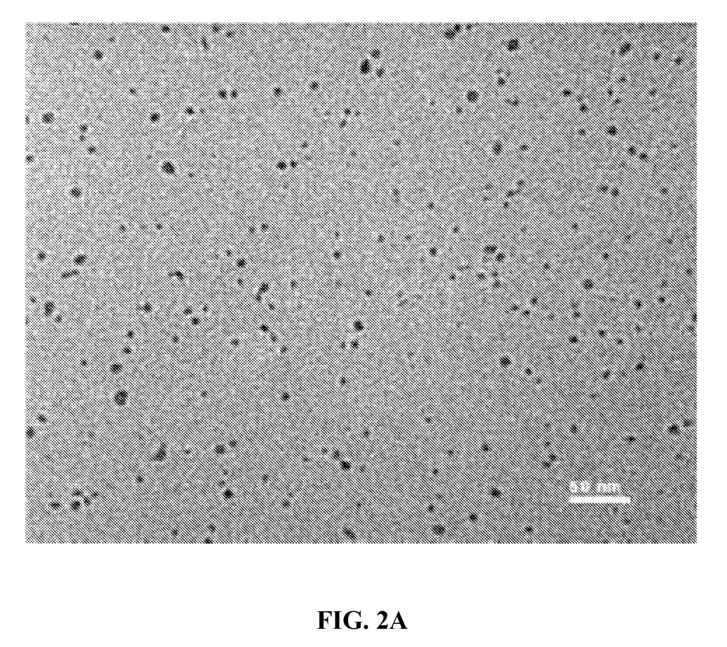

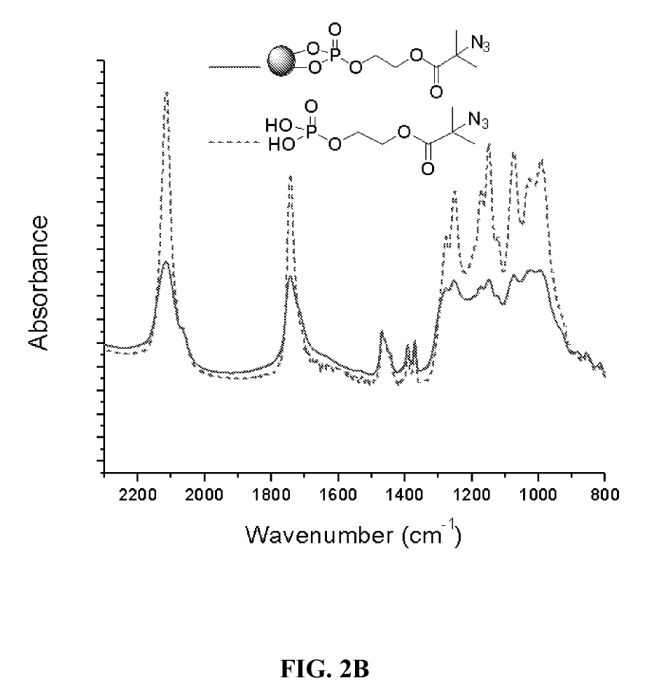

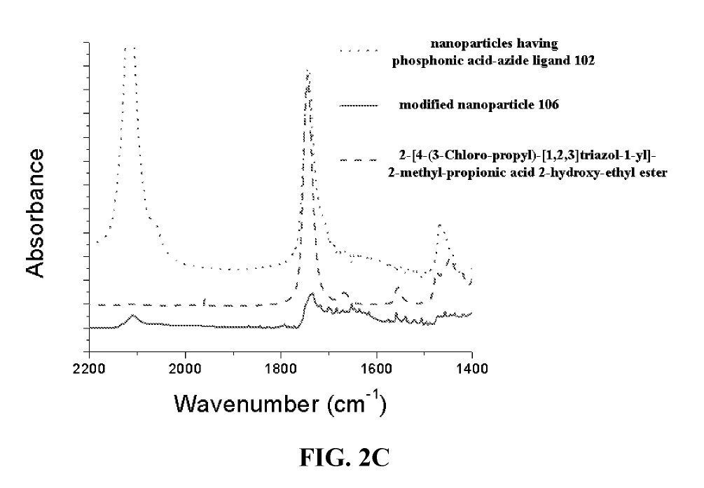

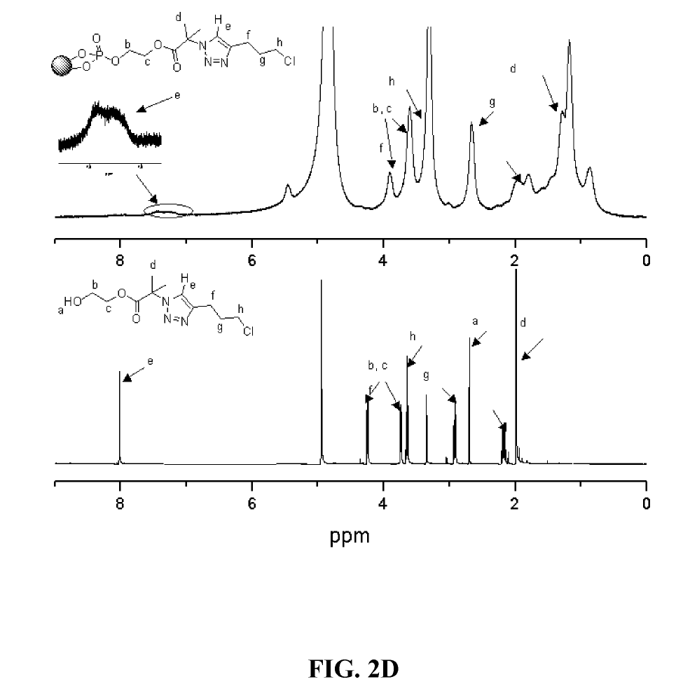

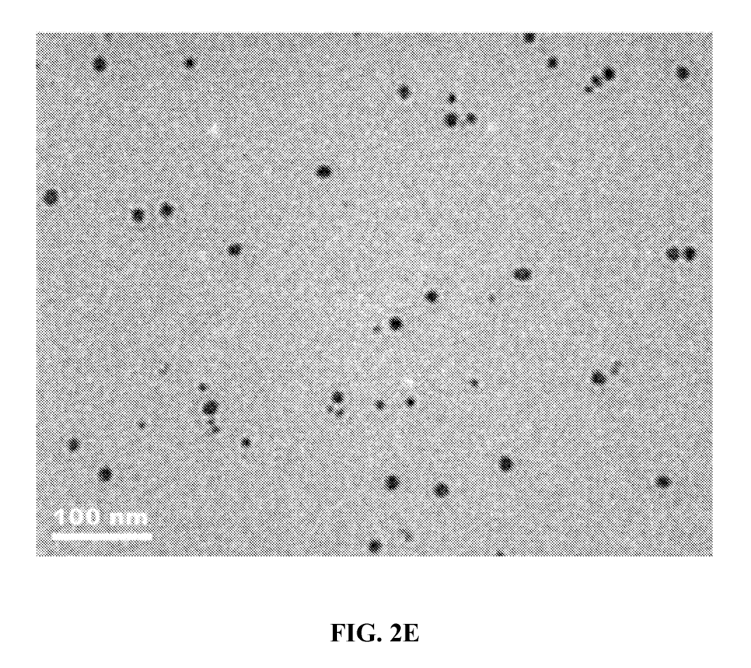

The oleic acid was stripped from the particles and exchanged with a phosphonic acid-azide ligand to obtain nanoparticles having phosphonic acid-azide ligand 102 (see FIG. 2) as follows. 10 mL of ethanol was added to a solution of oleic acid coated maghemite in CHCl₃ (5 mL). The solution became cloudy. The solution was then centrifuged, and the precipitated particles were collected. More ethanol was added to the solid particles (5 mL) and the solution was sonicated to break up the particle aggregates. The solution was then centrifuged and the precipitated particles were collected. This washing procedure was repeated twice. Then, a 1:1 weight ratio of the phosphonic acid ligand:Fe₂O₃ nanoparticles was added to a centrifuge tube. Approximately 5 mL of CHCl₃ was added to the particles. The resultant mixture was then sonicated for 10 minutes until the particles appeared to be dispersed. Hexane was then added to the solution of particles until the mixture became cloudy to remove excess phosphonic acid ligand that was not attached to the surface of the particles. The mixture was then centrifuged, and the precipitate was collected while the supernatant was discarded. The precipitated particles were then redispersed in CHCl₃. The particles were no longer soluble in hexane and other non-polar solvents.

Transmission electron microscope (TEM) images of the nanoparticles having phosphonic acid-azide ligand 102 indicated that they had not formed aggregates and that their size did not change upon ligand exchange within the limits of TEM accuracy. FIG. 2A shows a representative TEM image of the Fe₂O₃ nanoparticles coated with the phosphonic acid-azide ligand. As shown, there is no evidence of particle agglomeration, the nanoparticles are relatively monodisperse, and the sizes are on the order of about 10 nm.

Moreover, it was estimated from thermogravimetric analysis (TGA) data that the surface coverage of the particles was about 1.24±0.24 ligand/nm² based on a spherical model and particle diameter as estimated by TEM images.

As shown in FIG. 2B, Fourier transform infrared (FTIR) spectra of the nanoparticles having phosphonic acid-azide ligand 102 were compared to those of the free unbound phosphonic acid-azide ligand. The results showed that there was a very strong absorbance at 2114 cm⁻¹ due to the azide N═N═N antisymmetric stretch, as well as a strong absorbance at 1742 cm⁻¹ due to the C═O stretch of the ester. There was also a series of stretching bands from 1250-990 cm⁻¹ that can be assigned to the P—O and P═O stretches of the phosphonic acid group. As shown, the unbound phosphonic acid-azide ligand exhibits stronger absorbances demonstrating binding of the phosphonic acid-azide ligand to the nanoparticle.

A copper(I) catalyzed azide-alkyne cycloaddition (CuAAC) reaction using the complementary “click” functional molecule 5-chloropentyne 104 was performed to prepare a modified nanoparticle 106 (see FIG. 2). In a round bottom flask in air, nanoparticles having phosphonic acid-azide ligand 102 (30 mg, 0.103 mmol of phosphonic acid-azide ligand at the surface) was dissolved in 5 mL of 4:1 DMSO:H₂O. To this mixture was added 5-chloropentyne (13 μL, 0.124 mmol), followed by CuSO₄.5H₂O (3.1 mg, 0.008 mmol) and sodium ascorbate (4.1 mg, 0.0206 mmol). The reaction mixture was stirred in air at room temperature for 24 hours. 1 mL of CHCl₃, 1 mL of acetone, and 1 mL of ethanol were added to the mixture. The mixture was then centrifuged, and the precipitated nanoparticles were collected and characterized. The nanoparticles were soluble in methanol and chloroform, but not in less polar solvents. The nanoparticles were not soluble in H₂O.

Control reactions were also carried by reacting 2-azido-2-methyl-propionic acid 2-hydroxy-ethyl ester with 5-chloropentyne to obtain 2-[4-(3-chloro-propyl)-[1,2,3]triazol-1-yl]-2-methyl-propionic acid 2-hydroxy-ethyl ester.

As shown in FIG. 2C, the FTIR spectrum of the modified nanoparticle 106 and 2-[4-(3-chloro-propyl)-[1,2,3]triazol-1-yl]-2-methyl-propionic acid 2-hydroxy-ethyl ester both showed a loss of the N═N═N stretching band, indicating a high yield for the CuAAC reaction. Moreover, a peak at 1554 cm⁻¹ is observed agreeing with the literature value for a 1,2,3-triazole (see Billes, F., Endredi, H., Keresztury, G., J. Mol. Struct. 2000, vol. 530, p. 183, which is hereby incorporated by reference herein in its entirety). The absorbance bands due to the phosphonic acid group (1250-990 cm⁻¹) are still present, implying that the ligand is still attached to the particles and the phosphonate is still intact.

Very dilute samples of modified nanoparticle 106 were examined by proton nuclear magnetic resonance (¹H NMR) (see Willis, A. L., Turro, N. J., O'Brien, S., Chem. Mater. 2005, vol. 17, p. 5970). As shown in FIG. 2D, the characteristic peak at ˜8.0 ppm due to the triazole proton was clearly present.

FIG. 2E shows a TEM image of the modified nanoparticles.

The spectroscopic evidence from FTIR and NMR, coupled with the TEM images showing dispersed particles, suggest that the 1,3-dipolar cycloaddition was successful and that the particles are stabilized from aggregation by the new ligand system.

### Example 2

γ-Fe₂O₃ nanoparticles having oleic acid as a ligand were synthesized as described in Yin, M., Willis, A., Redl, F. Turro, N. J., O'Brien, S. P., J. Mater. Res. 2004, 19, 1208. The nanoparticles are crystalline and well dispersed and have about less than 5% rms variation in size.

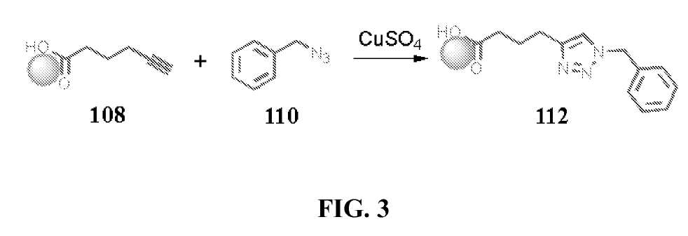

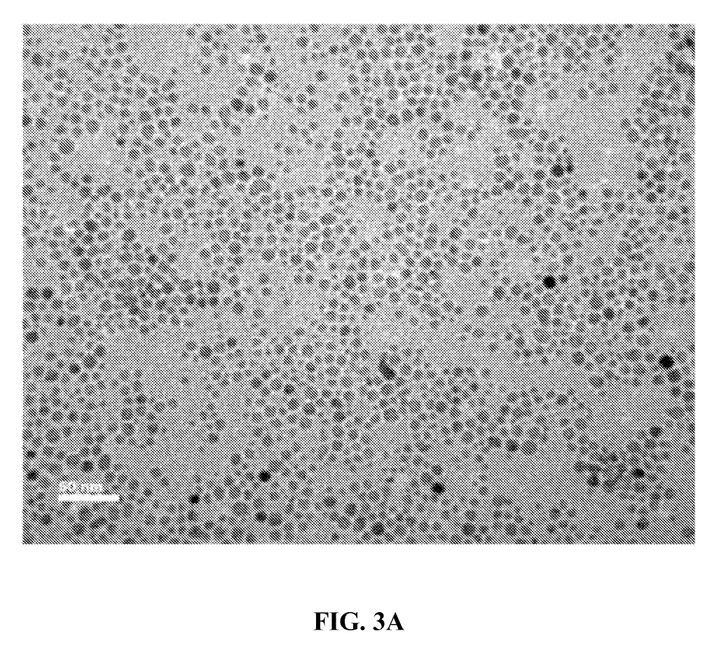

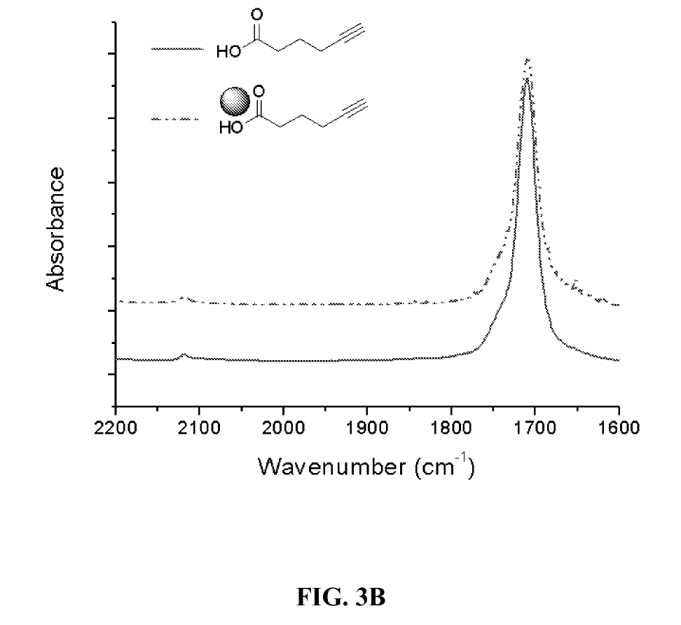

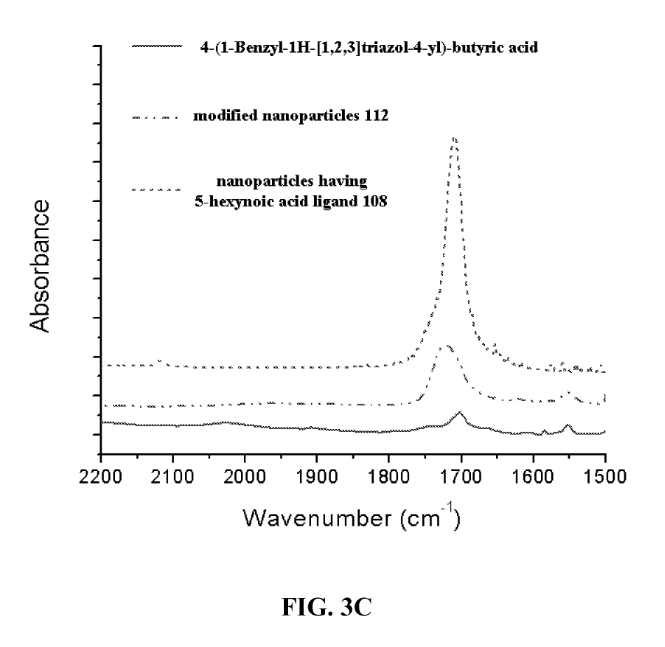

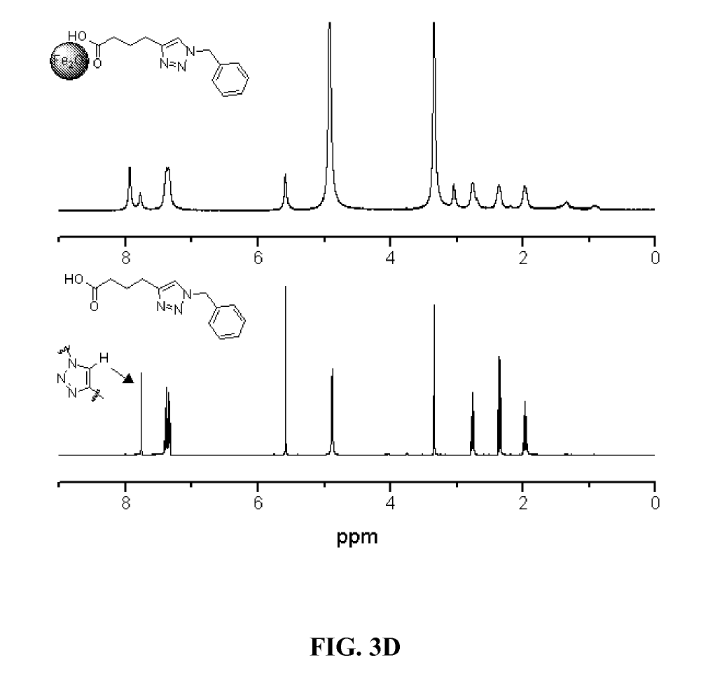

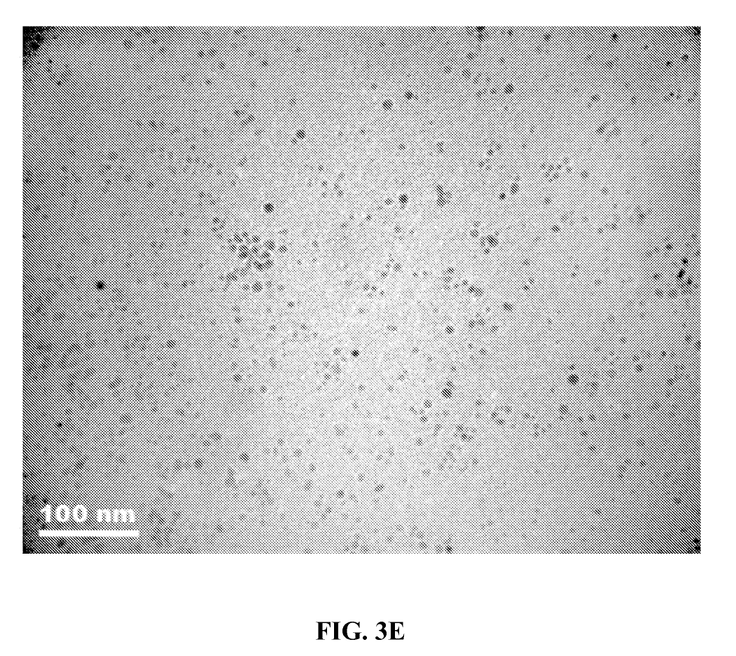

The oleic acid was stripped from the nanoparticles and exchanged with 5-hexynoic acid ligand (purchased from Aldrich) to obtain nanoparticles having 5-hexynoic acid ligand 108 (see FIG. 3) as follows. A 1:1 weight percent ratio of 5-hexynoic acid:Fe₂O₃ was added to a centrifuge tube. Approximately 5 mL of hexane was added to the nanoparticles. The resultant mixture was then sonicated for 20 minutes until the particles appeared to be dispersed. Ethanol was then added to the solution of particles until the mixture became cloudy to remove excess 5-hexynoic acid that was not attached to the surface of the particles. The mixture was then centrifuged, and the precipitate was collected while the supernatant was discarded. The precipitated particles were then redispersed in hexane.

TEM images of the nanoparticles having 5-hexynoic acid ligand 108 indicated that they had not formed aggregates and that their size did not change upon ligand exchange within the limits of TEM accuracy. FIG. 3A shows a representative TEM image of the Fe₂O₃ nanoparticles coated with the 5-hexynoic acid ligand. As shown, there is no evidence of particle agglomeration, the nanoparticles are relatively monodisperse, and the sizes are on the order of about 10 nm.

Moreover, it was estimated from thermogravimetric analysis (TGA) data that the surface coverage of the particles was about 11 ligand/nm² based on a spherical model and particle diameter as estimated by TEM images.

As shown in FIG. 3B, FTIR spectra of the nanoparticles having 5-hexynoic acid ligand 108 were compared to those of the free unbound 5-hexynoic acid ligand. In both samples, the results showed very weak absorbance peak at 2119 cm⁻¹ due to the alkyne as well as a strong absorbance at 1710 cm⁻¹ due to the C═O stretch of the carboxylic acid.

A copper(I) catalyzed azide-alkyne cycloaddition (CuAAC) reaction using a complementary “click” functional molecule, benzyl azide 110, was performed to prepare modified nanoparticles 112. In a round bottom flask in air, nanoparticles having 5-hexynoic acid ligand 108 (232 mg, 2.07 mmol of 5-hexynoic acid at the surface) were dissolved in 5 mL of 4:1 DMSO:H₂O. To this mixture was added benzyl azide (276 mg, 2.07 mmol), CuSO₄.5H₂O (62 mg, 0.25 mmol), and sodium ascorbate (81 mg, 0.41 mmol). The reaction was stirred overnight at room temperature in air. H₂O was added to precipitate the particles and centrifuged. The precipitate was collected and dispersed in a mixture of CHCl₃:ethanol (8:2).

Control reactions were also carried by reacting 5-hexynoic acid with benzyl azide to obtain 4-(1-benzyl-1H-[1,2,3]triazol-4-yl)-butyric acid.

As shown in FIG. 3C, the FTIR spectrum of modified nanoparticles 112 and 4-(1-benzyl-1H-[1,2,3]triazol-4-yl)-butyric acid shows a characteristic band at 1551 cm⁻¹ and no band at 2100 cm⁻¹, indicating conversion of the alkyne group to a triazole (see Billes, F., Endredi, H., Keresztury, G., J. Mol. Struct. 2000, vol. 530, p. 183, which is hereby incorporated by reference herein in its entirety).

Very dilute samples of modified nanoparticle 112 were examined by ¹H NMR (see Willis, A. L., Turro, N. J., O'Brien, S., Chem. Mater. 2005, vol. 17, p. 5970, which is hereby incorporated by reference herein in its entirety). As shown in FIG. 3D, the characteristic peak at ˜8.0 ppm due to the triazole proton was clearly present.

FIG. 3E shows a TEM image of the modified nanoparticles.

The spectroscopic evidence from FTIR and NMR, coupled with the TEM images showing dispersed particles, suggest that the 1,3-dipolar cycloaddition was successful and that the particles are stabilized from aggregation by the new ligand system.

### Example 3

γ-Fe₂O₃ nanoparticles having oleic acid as a ligand were synthesized as described in Yin, M., Willis, A., Redl, F. Turro, N. J., O'Brien, S. P., J. Mater. Res. 2004, 1 vol. 9, p. 1208. The nanoparticles are crystalline and well dispersed and have about less than 5% rms variation in size.

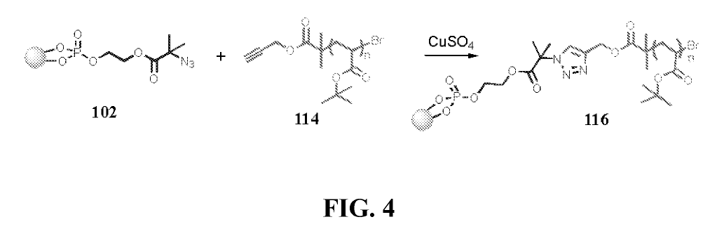

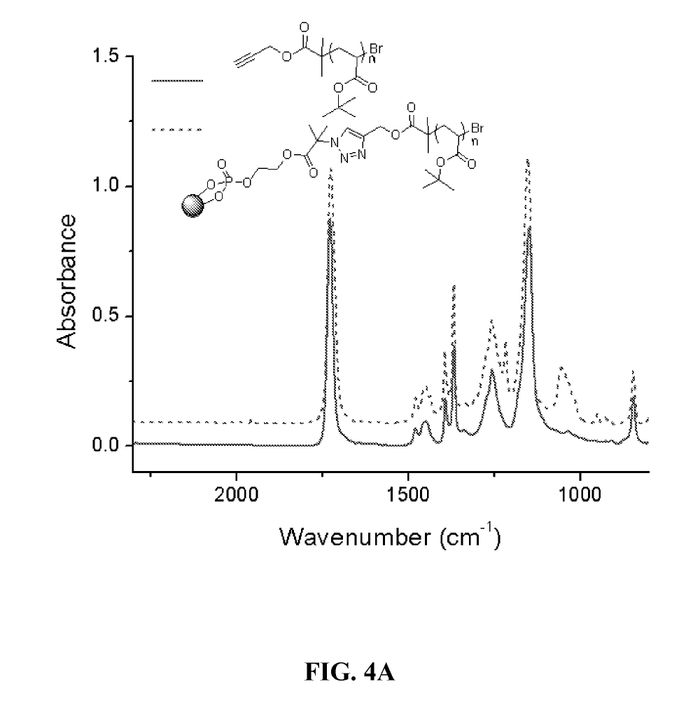

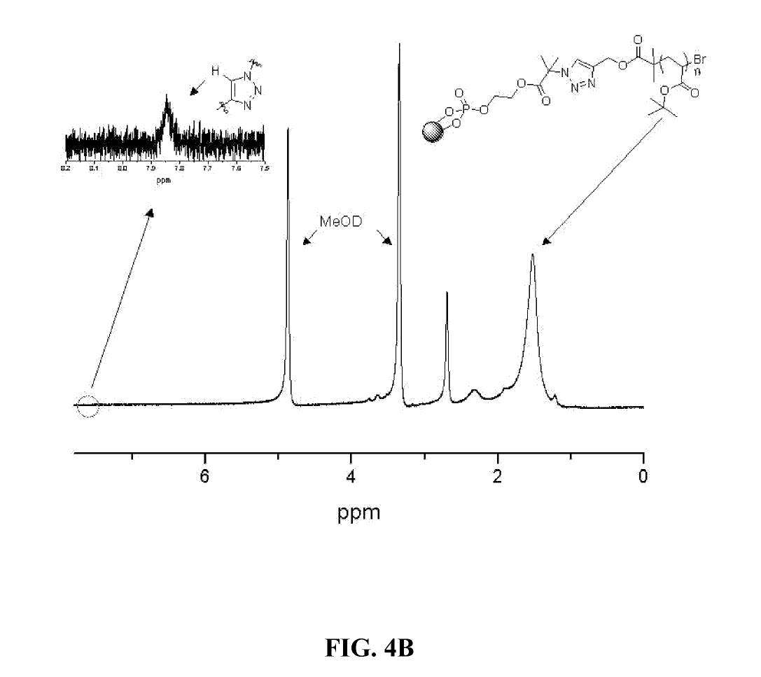

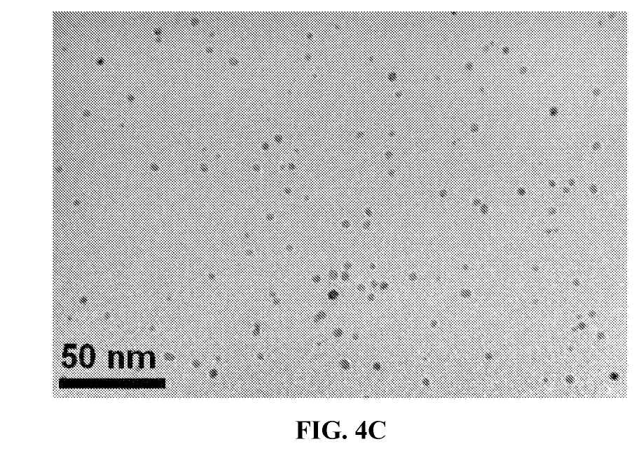

The oleic acid was stripped from the nanoparticles and exchanged with a phosphonic acid ligand 102 to obtain nanoparticles having phosphonic acid ligand 102 (see FIG. 4) as described above in EXAMPLE 1.

An α-acetylene-poly(tert-butyl acrylate) (ptBA) polymer 114 was prepared by atom transfer radical polymerization (ATRP) as follows: CuBr (168 mg, 1.17 mmol) and 2-propynyl 2-bromo-2-methylpropanoate (240 mg, 1.17 mmol) were added to a clean, dry round bottom flask, which was subsequently evacuated for 15 minutes and back-filled with argon. Freshly distilled tert-butyl acrylate (11.4 g, 88.9 mmol) was added via a degassed syringe followed by degassed toluene (5.7 mL), and PMDETA (1.95 g, 11.7 mmol). The reaction flask was immediately submerged in liquid N₂, and backfilled with argon. When the mixture thawed completely, the flask was submerged in a 70° C. oil bath and stirred for 12 hours under argon atmosphere. After this time, the reaction flask was opened to air, frozen in liquid nitrogen, thawed and diluted with tetrahydrofuran (20 mL). This solution was passed through a column of neutral alumina, concentrated on a rotary evaporator, precipitated in a 10:1 volume of 50-50 methanol-water:toluene three times, dissolved in diethyl ether, dried over MgSO₄, filtered, concentrated on a rotary evaporator, and dried under vacuum for two days to yield ptBA.

Based on the estimated surface coverage of the ligand on the nanoparticles, a 1:1 molar ratio of alkyne on the ptBA polymer 114 to azide on the phosphonic acid-azide ligand of nanoparticle 102 was determined. Based on this calculation, 64 mg (0.22 mmol) of nanoparticles having phosphonic acid-azide ligand 102 was dissolved in 4 mL DMSO in a round bottom flask in air. 1 mL of H₂O was then added. Then, 2.4 g (0.241 mmol) of ptBA 114 were added. Then, additional 35 mL of 4:1 DMSO:H₂O were added. Next, CuSO₄.5H₂O (6.7 mg, 0.027 mmol), sodium ascorbate (8.7 mg, 0.044 mmol), and BIPY (5.4 mg, 0.034 mmol) were added and heated to 60° C. in air for 36 hours. The reaction was stopped and chloroform was added with saturated solution of NH₄Cl. Extraction was then carried out for three times with chloroform and the organic extracts were washed with NH₄Cl solution to remove excess copper. The sample was then concentrated using rotary evaporator, followed by washing of the nanoparticles with 10:1 methanol:water mixture to precipitate the nanoparticles and remove excess unbound polymer. The solution was centrifuged, and the precipitated particles were collected (brown in color) and redispersed in THF.

The nanoparticles were characterized using TEM, FTIR, and NMR, as described above. As shown in FIG. 4A, the FTIR spectrum of the nanoparticles coated with ptBA 116 showed the disappearance of the azide peak at 2114 cm⁻¹, indicating high yield of the CuAAC reaction. The FTIR spectrum further contained characteristic peaks due to the ptBA, as well as the peaks around 1100 cm⁻¹ due to the phosphonic acid.

Furthermore, as shown in the ¹H NMR spectrum of FIG. 4B, the presence of the triazole proton at ˜8 ppm was also detected.

TEM images of the nanoparticles also revealed that they were well dispersed and that no aggregation had occurred (see FIG. 4C).

### Example 4

Titanium dioxide (TiO₂) nanoparticles having oleic acid ligands were synthesized as described in Maliakal, A., Katz, H., Cotts, P. M., Subramoney, S., Mirau, P. J. Am. Chem. Soc. 2005, vol. 127, p. 14655.

Dodec-11-ynyl-phosphonic acid diethyl ester (phosphonate-alkyne ligand) was synthesized as follows. In oven-dried glassware under nitrogen gas, 1,12-dibromodecane (15 g, 50.1 mmol) was dissolved in 20 mL anhydrous DMF. Sodium acetylide (14.7 g, 18 wt % in xylene and light mineral oil) was added drop wise to the reaction mixture. The mixture was then heated to 70° C. and stirred under nitrogen for 4 hours. A light precipitate was formed. An equal volume of water was added to the mixture, and the precipitate was dissolved. The mixture was extracted three times with chloroform and the combined organic extracts were washed five times to remove DMF. The organics were dried with MgSO₄, filtered, and the solvent was removed by rotary evaporator to obtain 12-bromo-dodec-1-yne. In oven-dried glassware under nitrogen, 12-bromo-dodec-1-yne (12.3 g, 50.1 mmol) was added to triethyl phosphate (21.8 mL, 125.25 mmol). The mixture was heated to 150° C. and stirred for 17 hours. The mixture was allowed to cool to room temperature, and the excess triethyl phosphite was removed under vacuum. The resultant amber liquid was purified by flash column chromatography with a solvent gradient from 0-75% EtOAC:hexane and the phosphonate-alkyne ligand was isolated as a light yellow liquid (1.8 g, 18% overall).

The oleic acid was stripped from the nanoparticles and exchanged with phosphonate-alkyne ligand as follows. The number of surface groups was estimated using a rod model for the nanoparticle surface area calculation. The oleic acid coated titania nanoparticles (660 mg, 1.58 mmol) were added to a dry round bottom flask under nitrogen and dispersed in 15 mL of chlorobenzene. Dodec-11-ynyl-phosphonic acid diethyl ester was then added, and the resultant mixture was heated to 100° C. for 48 h. A transparent yellow solution resulted. Methanol was added to the solution to precipitate the particles and the mixture was then centrifuged. The precipitate was collected and the supernatant was discarded. Methanol was then added to the precipitate and the mixture was sonicated and centrifuged. The supernatant was discarded and the precipitate was washed in this manner two more times to remove oleic acid and unbound ligand. The resultant particles were dispersible in at least CHCl₃, CH₂Cl₂, and chlorobenzene.

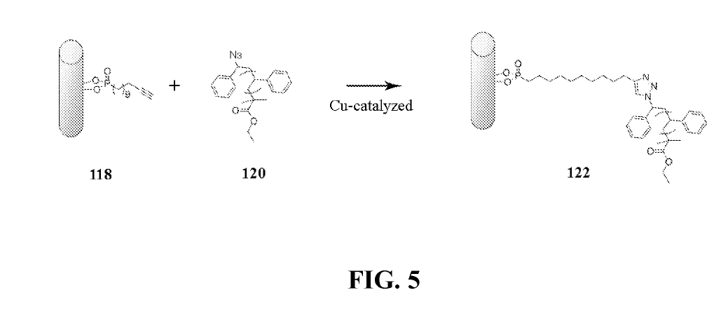

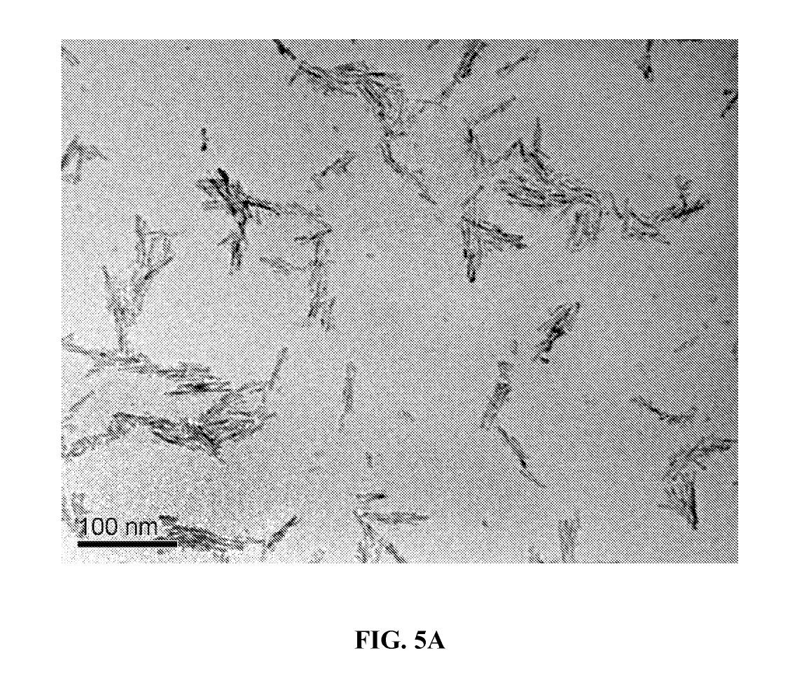

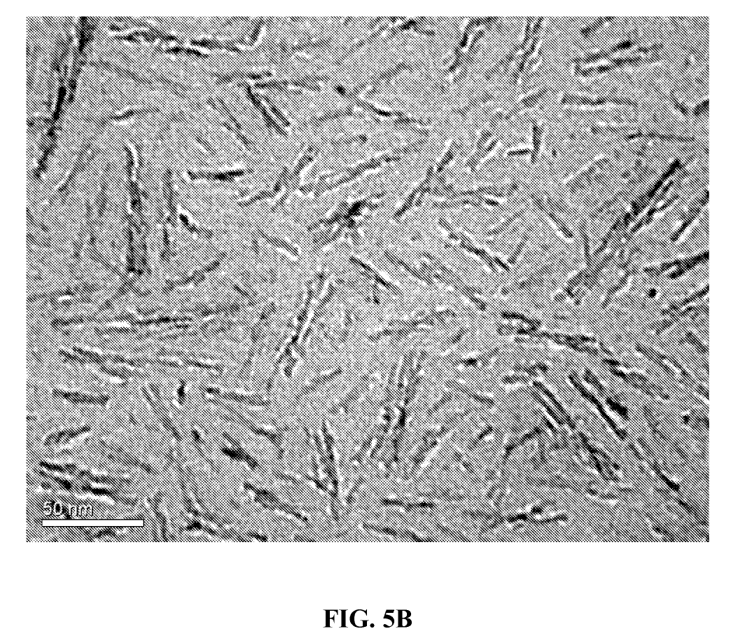

The nanoparticles functionalized with the phosphonate-alkyne ligand 118 were examined using TEM and FTIR. The FTIR spectra (not shown) showed the presence of the characteristic peaks of a mono-substituted alkyne: 3315 cm⁻¹ and 2120 cm⁻¹ for the C—H and C—C triple bond stretches, respectively. FIG. 5A is a representative TEM image showing the titania nanoparticles functionalized with the phosphonate-alkyne ligand 118.

To carry out a click chemistry, ω-azido polystyrene 120 [Mₙ=8093, PDI=1.18] was synthesized as follows. Styrene was passed through an alumina column to remove polymerization inhibitors. Styrene (27 mL, 234 mmol) and PMDETA (0.97 mL, 4.68 mmol) were added to a dry round bottom flask. The mixture was degassed by bubbling with nitrogen gas for 45 minutes. CuBr (671 mg, 4.68 mmol) and ethyl α-bromoisobutyrate (0.7 mL, 4.68 mmol) were added to the mixture under nitrogen gas. The reaction vessel was placed in a 100° C. oil bath and allowed to stir for 20 hours. The resulting ω-bromo-polystyrene was dissolved in CH₂Cl₂ and passed through a column of alumina to remove residual copper. The solvent was removed using a rotary evaporator. Then, ω-bromo-polystyrene (4.68 mmol) was dissolved in 60 mL of DMF under nitrogen gas. Sodium azide (5.62 mmol) was added and the mixture was heated to 60° C. in an oil bath for 48 hours. The resulting ω-azido polystyrene 120 was dissolved in CHCl₃, washed with H₂O, dried over MgSO₄, and filtered. The solvent was removed by a rotary evaporator.

TiO₂ nanoparticles having ω-azido polystyrene 122 were obtained as follows (see FIG. 5). Nanoparticles functionalized with the phosphonate-alkyne ligand 118 (200 mg, 0.245 mmol) were dispersed in 5 mL chlorobenzene. ω-azido polystyrene (0.245 mmol) was added to the solution under nitrogen gas. [(CH₃CN)₄Cu]PF₆ (18 mg, 0.049 mmol) was added to the mixture. The reaction was then heated to 100° C. for 48 hours. After the reaction was allowed to cool to room temperature, methanol was added to precipitate the particles. The mixture was then centrifuged and the precipitate was collected while the supernatant was discarded. 9:1 THF:methanol was then added to the precipitate and the mixture was sonicated and centrifuged. The supernatant was discarded and the precipitate was washed in this manner two more times to wash away unbound polymer.

FIG. 5B shows a representative TEM image of TiO₂ nanoparticles having ω-azido polystyrene 122.

### Example 5

TiO₂ nanoparticles having oleic acid as a ligand were synthesized as described in Maliakal, A., Katz, H., Cotts, P. M., Subramoney, S., Mirau, P. J. Am. Chem. Soc. 2005, vol. 127, p. 14655.

Dodec-11-ynyl-phosphonic acid diethyl ester (phosphonate-alkyne ligand) was synthesized as described in EXAMPLE 4.

The oleic acid was stripped from the nanoparticles and exchanged with phosphonate-alkyne ligand as described in EXAMPLE 4.

To carry out click chemistry, ω-azido poly(tert-butyl acrylate) 124 [Mₙ=6483, PDI=1.05] was synthesized as follows. Tert-butyl acrylate (tBA) was passed through an alumina column prior to reaction in order to remove inhibitor. The tBA (17.0 mL, 117.03 mmol), PMDETA (0.27 mL, 1.29 mmol), and acetone (4 mL) was added to a dry round bottom flask. The mixture was degassed by bubbling with nitrogen gas for 45 minutes. CuBr (168 mg, 1.17 mmol) and ethyl α-bromoisobutyrate (0.35 mL, 2.34 mmol) were added to the mixture under nitrogen gas and the vessel was placed in a 60° C. oil bath. Then, the reaction was stirred for 28 hours. The mixture was opened to air and diluted with acetone. Subsequently, the solution was passed through a column of alumina to remove any excess copper and the solvent was removed on a rotary evaporator. The resulting ω-bromo poly(tert-butyl acrylate) was obtained as a viscous material. Then, ω-bromo poly(tert-butyl acrylate) (4.68 mmol) was dissolved in 60 mL of DMF under nitrogen gas. Sodium azide (5.62 mmol) was added and the mixture was heated to 60° C. in an oil bath for 48 hours to obtain ω-azido poly(tert-butyl acrylate). The resulting ω-azido poly(tert-butyl acrylate) was dissolved in CHCl₃, washed with H₂O, dried over MgSO₄, and filtered. The solvent was removed by a rotary evaporator.

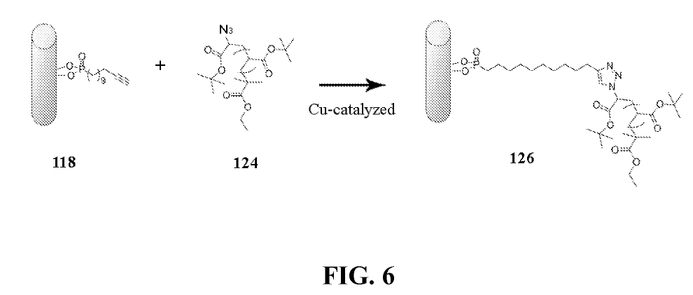

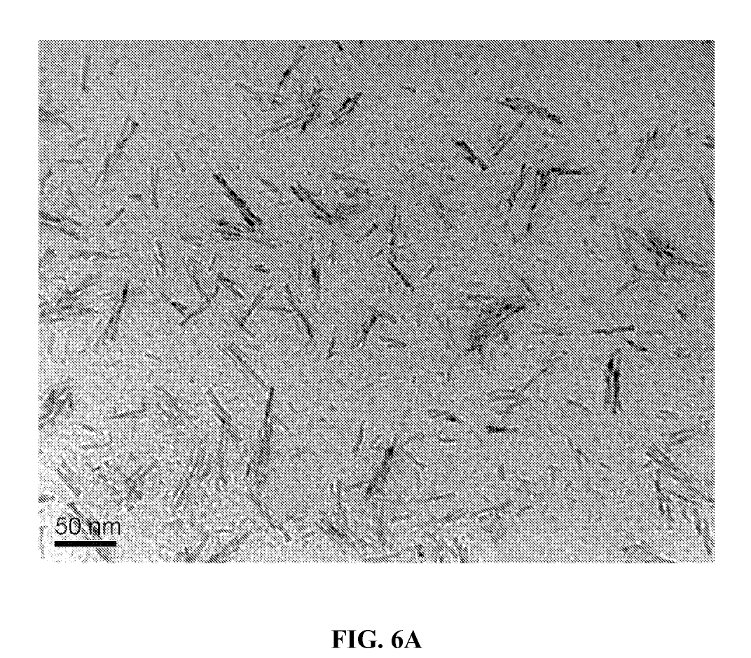

TiO₂ nanoparticles having ω-azido poly(tert-butyl acrylate) 126 were obtained as follows (see FIG. 6). Nanoparticles functionalized with the phosphonate-alkyne ligand 118 (200 mg, 0.245 mmol) was dispersed in 5 mL chlorobenzene. ω-azido poly(tert-butyl acrylate) (0.245 mmol) was added to the solution under nitrogen gas. [(CH₃CN)₄Cu]PF₆ (18 mg, 0.049 mmol) was added to the mixture. The reaction was then heated to 100° C. for 48 hours. After the reaction was allowed to cool to room temperature, methanol was added to precipitate the particles. The mixture was then centrifuged and the precipitate was collected while the supernatant was discarded. Methanol was then added to the precipitate and the mixture was sonicated and centrifuged. The supernatant was discarded and the precipitate was washed in this manner two more times to wash away any unbound polymer.

FIG. 6A shows a representative TEM image of TiO₂ nanoparticles having ω-azido poly(tert-butyl acrylate) 126.

### Example 6

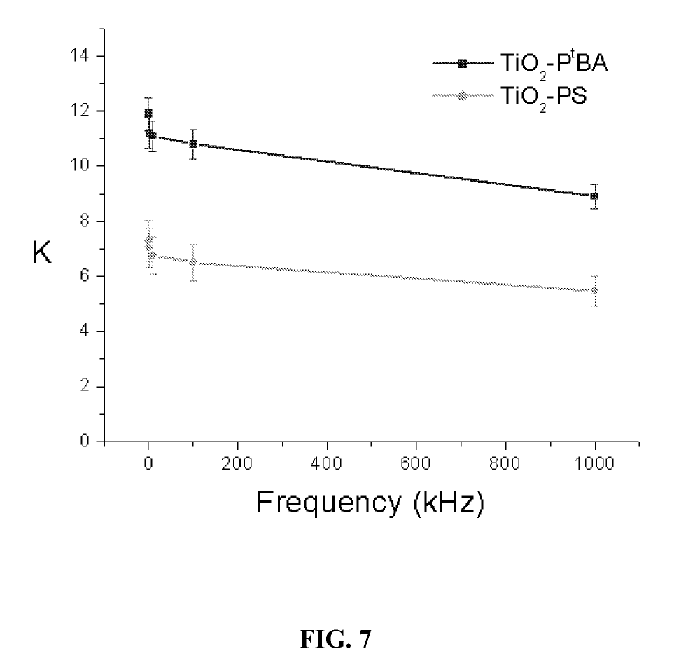

FIG. 7 shows dielectric measurements for 0.3 μm thick TiO₂ nanoparticles having ω-azido polystyrene 122 (TiO₂—PS) and 0.08 μm TiO₂ nanoparticles having ω-azido poly(tert-butyl acrylate) 126 films. As shown, film having high dielectric constants can be obtained.

Upon review of the description, embodiments, and examples of the invention described above, those skilled in the art will understand that modifications and equivalent substitutions can be performed in carrying out the invention without departing from the essence of the invention. Thus, the invention is not meant to be limiting by the embodiments described explicitly above, and is limited only by the claims which follow.

What is claimed is:

1. A method for controlling the surface functionality of metal oxide nanoparticle, the method comprising:
attaching a ligand to metal oxide nanoparticle, the ligand comprising a functional portion that is capable of forming an irreversible bond with an object at one or more reactive sites that are complementary to the functional portion without reacting with other reactive sites that may be present.

2. The method of claim 1, further comprising:
reacting the ligand with the object.

3. The method of claim 1, further comprising:
reacting the ligand with the object using a click chemistry.

4. The method of claim 3, wherein the functional portion comprises an azide or an alkyne group.

5. The method of claim 4, wherein the reacting comprises adding a copper catalyst to carry out an azide-alkyne cycloaddition.

6. The method of claim 1, wherein the ligand further comprises an anchoring portion that attaches to the metal oxide nanoparticle, the anchoring portion being selected from the group consisting of carboxylates, alcohols, phosphonates, phosphonic acid esters, siloxanes, enediols, diols, and catechols.

7. The method of claim 1, wherein the ligand is selected from the group consisting of trioctylphosphine oxide, oleic acid, myristic acid, caprylic acid, 2-bromo-2-methylpropionic acid, dodecanol, 2,2′-didodecyl-1,3-dihydroxypropane, 2-dodecyl-1,3-dihydroxypropane, tridodecylcarbinol, didodecylcarbinol, octadecylphosphonic acid, lauric acid-liposome, trioctylamine, octylamine, dodecylamine, hexadecylamine, oleylamine, octanethiol, and dodecanethiol.

8. The method of claim 1, wherein the metal oxide nanoparticle is selected from the group consisting of iron oxide, titanium oxide, silicon oxide, aluminum oxide, vanadium oxide, copper oxide, cobalt oxide, manganese oxide, zinc oxide, europium oxide, gadolinium oxide, indium oxide, barium titanium oxide, manganese iron oxide, cobalt iron oxide, nickel iron oxide, zinc iron oxide, and mixtures thereof.

9. The method of claim 1, wherein the metal oxide nanoparticles are greater than approximately 1 nm and less than approximately 1000 nm.

10. The method of claim 9, wherein the metal oxide nanoparticles are greater than approximately 10 nm and less than approximately 100 nm.

11. The method of claim 1, wherein the metal oxide nanoparticles are spherical, rod-like, plate-like, ellipsoidal, hemispherical, hemiellipsoidal, tripod-like, or tetrapod-like in shape.

12. The method of claim 1, wherein the ligand is 2-azido-2-methyl-propionic acid 2-phosphonooxy-ethyl ester or 5-hexynoic acid and the nanoparticle is iron oxide.

13. The method of claim 1, wherein the ligand is dodec-11-ynyl-phosphonic acid diethyl ester and the nanoparticle is titanium dioxide.

14. A metal oxide nanoparticle comprising:
a ligand which comprises
an anchoring portion that attaches to a surface of the metal oxide nanoparticle; and
a functional portion that is capable of forming an irreversible bond with an object at one or more reactive sites that are complementary to the functional portion without reacting with other reactive sites that may be present.

15. The metal oxide nanoparticle of claim 14, further comprising:
the object bonded with the functional portion of the ligand.

16. The metal oxide nanoparticle of claim 15, wherein
the object was bonded with the functional portion of the ligand via click chemistry.

17. The metal oxide nanoparticle of claim 15, wherein the functional portion comprises an azide or an alkyne group.

18. The metal oxide nanoparticle of claim 17, wherein the complementary site comprises an alkyne group when the functional portion comprises an azide group or the complementary site comprises an azide group when the function portion comprises an alkyne group.

19. The metal oxide nanoparticle of claim 14, wherein the anchoring portion is selected from the group consisting of carboxylates, alcohols, phosphonates, phosphonic acid esters, siloxanes, enediols, diols, and catechols.

20. The metal oxide nanoparticle of claim 14, wherein the ligand is selected from the group consisting of trioctylphosphine oxide, oleic acid, myristic acid, caprylic acid, 2-bromo-2-methylpropionic acid, dodecanol, 2,2′-didodecyl-1,3-dihydroxypropane, 2-dodecyl-1,3-dihydroxypropane, tridodecylcarbinol, didodecylcarbinol, octadecylphosphonic acid, lauric acid-liposome, trioctylamine, octylamine, dodecylamine, hexadecylamine, oleylamine, octanethiol, and dodecanethiol.

21. The metal oxide nanoparticle of claim 14, wherein the metal oxide nanoparticle is selected from the group consisting of iron oxide, titanium oxide, silicon oxide, aluminum oxide, vanadium oxide, copper oxide, cobalt oxide, manganese oxide, zinc oxide, europium oxide, gadolinium oxide, indium oxide, barium titanium oxide, manganese iron oxide, cobalt iron oxide, nickel iron oxide, zinc iron oxide and mixtures thereof.

22. The metal oxide nanoparticle of claim 14, wherein the metal oxide nanoparticles are greater than approximately 1 nm and less than approximately 1000 nm.

23. The metal oxide nanoparticle of claim 22, wherein the metal oxide nanoparticles are greater than approximately 10 nm and less than approximately 100 nm.

24. The metal oxide nanoparticle of claim 14, wherein the metal oxide nanoparticles are spherical, rod-like, plate-like, ellipsoidal, hemispherical, hemiellipsoidal, tripod-like, or tetrapod-like in shape.

25. The metal oxide nanoparticle of claim 14, wherein the ligand is 2-azido-2-methyl-propionic acid 2-phosphonooxy-ethyl ester or 5-hexynoic acid and the nanoparticle is iron oxide.

26. The metal oxide nanoparticle of claim 14, wherein the ligand is dodec-11-ynyl-phosphonic acid diethyl ester and the nanoparticle is titanium dioxide.

27. A method for treating cancer, the method comprising:
attaching a ligand to a metal oxide nanoparticle, the ligand comprising a functional portion that is capable of forming an irreversible bond with an marker that has an affinity to cancer cells at one or more reactive sites that are complementary to the functional portion without reacting with other reactive sites that may be present;
reacting the ligand with the marker to form a metal oxide nanoparticle having affinity to cancer cells;
administering a sufficient quantity of the nanoparticle having affinity to cancer cells to a patient in need thereof, and
detecting the cancer cells through magnetic resonance imaging.

25. The method of claim 24, wherein the metal oxide nanoparticle comprises an iron oxide nanoparticle.

26. The method of claim 25, wherein the marker comprises anti-vascular endothelial growth factor.

27. The method of claim 26, further comprising carrying out magnetic heating to kill the cancerous cells.

28. A method for forming a dielectric material in an electronic device, the method comprising:
attaching a ligand to a metal oxide nanoparticle having a dielectric constant that is at least 2, the ligand comprising a functional portion that is capable of forming an irreversible bond with a resin that is compatible with electronic device manufacturing requirements at one or more reactive sites that are complementary to the functional portion without reacting with other reactive sites that may be present;
reacting the ligand with the resin to form a metal oxide nanoparticle that is compatible with electronic device manufacturing requirements; and
depositing the metal oxide nanoparticle that is compatible with electronic device manufacturing requirements onto at least a portion of an electronic device.

29. The method of claim 28, wherein the dielectric constant is from about 30 to about 100.

30. The method of claim 28, wherein the metal oxide nanoparticle comprises titanium dioxide nanoparticle.

29. The method of claim 28, further comprising:
pattering the metal oxide nanoparticle that is compatible with electronic device manufacturing requirements using lithography.

30. The method of claim 28, wherein the depositing is carried out by printing.

31. A method for delivering a drug, the method comprising:
attaching a ligand to a metal oxide nanoparticle, the ligand comprising a functional portion that is capable of forming an irreversible bond with a block copolymer at one or more reactive sites that are complementary to the functional portion without reacting with other reactive sites that may be present;
reacting the ligand with the block copolymer to form a metal oxide nanoparticle;
incorporating the drug with the block copolymer;
administering a sufficient quantity of the nanoparticle having the block copolymer and the drug to a patient in need thereof.

32. The method of claim 31, wherein the block copolymer comprises at least one block which is hydrophilic and at least one block which is hydrophobic.

33. The method of claim 32, wherein the hydrophilic block is biocompatible and the hydrophobic block is capable is carrying a drug.

34. A composition comprising metal oxide nanoparticles, the metal oxide nanoparticles comprising:
a ligand which comprises
an anchoring portion that attaches to a surface of the metal oxide nanoparticle; and
a functional portion that is capable of forming an irreversible bond with a hydrophilic molecule at one or more reactive sites that are complementary to the functional portion without reacting with other reactive sites that may be present.

35. The composition of claim 34, wherein the metal oxide nanoparticles are capable of absorbing at least some ultraviolet radiation.

36. The composition of claim 35, wherein the composition is included in a sunscreen.

37. The composition of claim 35, wherein the composition is included in a paint formulation.
